(19) United States
(12) Patent Application Publication
(10) Pub. No.: US 2021/0403958 A1
(43) Pub. Date: Dec. 30, 2021

(54) SYSTEM AND METHOD FOR PRODUCING FUEL GRADE ETHANOL FROM CELLULOSIC AND HIGH STARCH COMBINED FEEDSTOCKS

(71) Applicant: Chemtex Global Corporation, Wilmington, NC (US)

(72) Inventors: Wenku Bill Xi, Wilmington, NC (US); Jeffrey E. Taylor, Wilmington, NC (US)

(73) Assignee: Chemtex Global Corporation, Wilmington, NC (US)

(21) Appl. No.: 17/359,785

(22) Filed: Jun. 28, 2021

Related U.S. Application Data
(60) Provisional application No. 63/045,408, filed on Jun. 29, 2020.

(51) Int. Cl. C12P 7/10

(52) U.S. Cl. CPC C12P 7/10; C12P 2201/00

(57) ABSTRACT

Ethanol is produced by the simultaneous production of both First and Second generation (1G, 2G) fuel grade ethanol in the same production plant. A First Generation feedstock such as corn is continuously fed to the first generation section and a lignocellulosic feedstock such as corn stover from the 1G corn is supplied to the second generation area Thus, there is a common fermentation area for both the C5 and C6 sugar fermentation. The invention can economically be best implemented in places where there are incentives offered for the use of various feedstocks. Specifically, the invention allows the D3 rin to be maximized in an existing first-generation ethanol plant with the installation of the front end of the 2G equipment.

### FIELD OF THE INVENTION

The invention relates generally to the production of fuel grade ethanol from a combination of cellulosic feedstock (e.g., corn stover) and a high starch feedstock (e.g., corn). Processes in accordance with the invention can utilize the same fermentation, distillation, and dehydration equipment of a conventional first generation ethanol plant.

### BACKGROUND

First generation (1G) ethanol plants employ high starch materials such as corn as a feedstock. Harvesting and separating the corn feedstock produces corn stover as a waste byproduct. The corn is milled, subjected to saccharification, typically with selected enzymes, and then fermented with selected yeast to produce ethyl alcohol. The ethyl alcohol is then concentrated, typically by distillation, to the selected alcohol percentage and stored for use. This process tends to be relatively energy intensive. However, not only is ethanol useful on its own, but corn-based ethanol can be a government backed renewable fuel and ethanol is used as an oxygenate in fuel blends to replace a prior carcinogen (MTBE). Also, certain government subsidies can exist that can make ethanol more economically competitive, compared to crude oil.

Second generation (2G) processes have developed to utilize the potential for processing the corn stover byproduct and other cellulosic materials into ethanol. Corn stover is a cellulosic feedstock and comprises the leaves, stalks, and cobs of the corn plants after harvest. Some reports indicate that stover may make up about half of the yield of a corn crop. Corn stover is similar to straw from other cereal grasses. These 2G processes employ certain enzymatic or other processes to convert the stover into fermentable sugars. 2G processes have also tended to be energy intensive. New technologies are emerging for stand-alone 2G facilities that are quite promising, in an effort to overcome some of the pitfalls of the earlier technologies.

However, existing 2G systems and processes have not proved to be fully satisfactory for widespread implementation.

When oil prices drop, the production of ethanol from corn becomes even less competitive. Therefore, during periods of low oil prices and/or low demand, the demand for corn produced ethanol becomes reduced and under certain circumstances, production at ethanol plants can be temporarily suspended.

Accordingly, it is desirable to develop alternative ethanol production systems and methods that overcome shortcomings of the prior art.

### SUMMARY OF THE INVENTION

Generally speaking, in accordance with the invention, a new system and method for producing ethanol is provided. The system and method can utilize various types of cellulosic feedstocks, such as corn stover, in an efficient manner. The system and method involve producing fuel ethanol from multiple feedstocks at the same time using common fermentation, distillation, dehydration and storage sections. Thus, in one embodiment of the invention, a 1G plant and process can be used to produce ethanol, e.g., from C6 sugars and the front portion (pretreatment, hydrolysis, lignin separation) of a 2G process can produce C5 and C6 sugars and proceed up to the fermentation step. At this point, the sugars from the 1G and 2G processes are combined and fermented together. Microorganisms selected to optimize fermentation of C5 sugars to alcohol are advantageously included. Accordingly, the front end area can be specific for each type of feedstock and then production joins at the fermentation step. In one embodiment of the invention, an existing 1G ethanol plant is joined with the front end of a 2G process, up to fermentation, either on site or off site and the sugars from both processes are combined for fermentation. Systems and methods in accordance with preferred embodiments of the invention can employ existing ethanol plants that may be idle during certain economic conditions.

Significant aspects of the invention are the use of a common plant from the fermentor through the distillation sections and storage for both the 1G and 2G processes. Preferred embodiments of the invention utilize the pretreatment area of the 2G area to breakdown the cellulosic material, using on site enzymes. Once the enzymes further breakdown the cellulosic and hemi cellulosic sugars to fermentable sugars, the sugars from this 2G process are combined with the sugars from accompanying 1G processes area in a common fermenter using onsite produced yeast optimized to ferment the combination. The starch from the corn in the 1G area is converted to sugars and the 1G sugars are pumped to the same common fermenter. The fermenters will ferment the 1G sugars and at the same time ferment the C5 and C6 sugars from the concomitant 2G process. Once fermented, the combined ethanol can be purified in a single distillation area and dehydration section and then pumped to a common storage area. By utilizing the agricultural crop residue and transforming this residual to ethanol it eliminates the need to burn off the excess residue in the field causing air quality concerns.

### BRIEF DESCRIPTION OF THE DRAWINGS

For a fuller understanding of the invention, reference is had to the following description, taken in connection with the accompanying drawing, in which:

FIG. 1 is a schematic flow chart of a combination 1G/2G integration approach to the manufacture of ethanol including low starch feedstock.

The FIGURE is for illustration only and should not be interpreted as limiting.

### DETAILED DESCRIPTION OF THE PREFERRED EMBODIMENTS

Manufacturing schemes in accordance with the invention include retrofitting of an existing 1G ethanol plant by joining a modified front-end pretreatment and hydrolysis section of a second generation (2G) process to allow usage of corn stover. This can provide an effective way to convert sugars in cellulosic feedstocks to high quality alcohol. Moreover, as a cellulosic feedstock, corn stover can receive approximately $1.50 per gallon D3 rin. Thus, not only are these combination plants an effective way to use material that is often discarded or burned, due to its low starch content, combination 1G/2G plants can remain economically viable even when stand alone 1G and 2G plants are not.

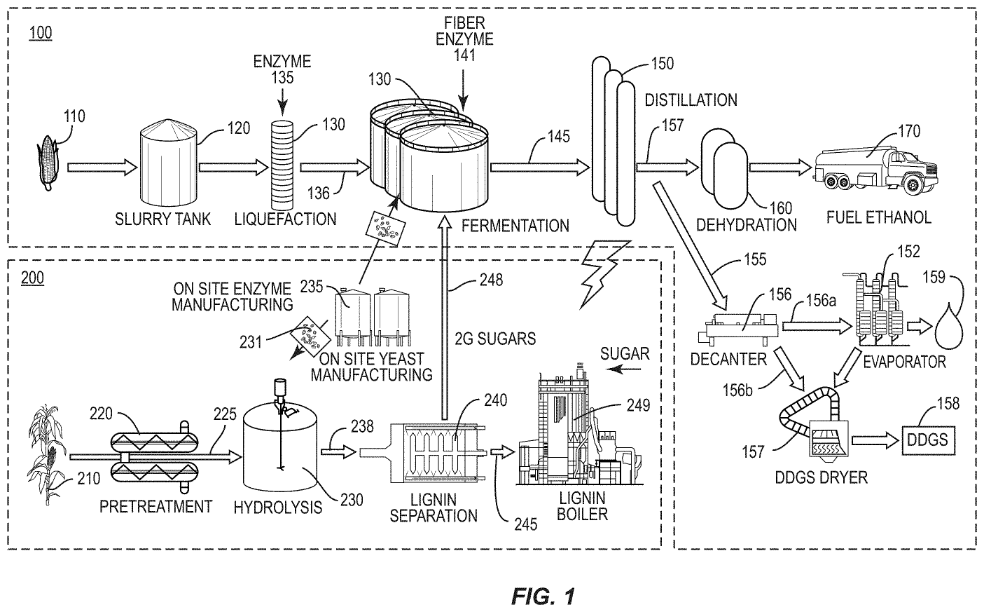

The corn rate can be reduced to maximize the second-generation sugars production to fully realize the D3 rin and operate within the constraints of the existing first-generation facility. In a preferred embodiment of the invention the equipment can be installed as depicted in FIG. 1 (below) in an existing, e.g., 100,000,000 gallon per year corn ethanol facility.
a. Congress created the renewable fuel standard (RFS) program, which the U.S. Environmental Protection Agency implements under consultation with the U.S. Department of Energy, to reduce greenhouse gas (GHG) emissions and expand the nation's renewable fuels sector while reducing reliance on imported oil. Environmental credits, called Renewable Identification Numbers (RINs or rins) are issued.
b. There is interest in producing fuel ethanol from lignocellulosic feedstocks such as corn stover. This reduces waste and increases the overall production from a given amount of land.
c. A process is provided herein for producing a fermentation formed product such as ethanol from lignocellulosic feedstocks. A pretreatment process is performed on the stover, followed by enzymatic hydrolysis of the cellulose into fermentable sugars. The pretreatment process disrupts the fiber structure of the feedstock to make it accessible to the enzymes. The pretreatment process opens the tight structure of the lignin to the hemi cellulose and cellulose in the feedstock. By breaking this tight structure, the cellulose and hemicellulose become accessible to the enzymes. The enzymes further breakdown the cellulose and hemicellulose into fermentable sugars.
d. One advantage of the processes and systems disclosed herein is to maximize the d3 rin. The yeast can be processed on site and should be optimized so that the same yeast component can be used to ferment the combined sugar streams simultaneously in the common fermenters.
e. Another advantage of processes and systems in accordance with the invention is that it permits more flexible use of the current equipment in an existing 1G ethanol plant. It is possible to only install the pretreatment and hydrolysis sections of a 2G plant along with the biomass boiler and on-site yeast and enzyme production areas. Operating the plant to maximize operating the 2G area at maximum throughput and reducing the throughput of the first-generation area to stay within the limits of the currently installed distillation area is preferred.
f. The lignin separated in the 2G area can be burned in a boiler or otherwise to make some or all of the steam required for the 2G area and also the steam for the 1G area. Productively burning the lignin can result in no excess lignin on site. This saves the natural gas or other fuel source currently used in typical plants for steam production and provides an efficient way to reduce waste that would have been produced from growing corn or wheat used in a conventional 1G process.
g. The fermentation conditions for both first generation and second generation ethanol are similar. One method in accordance with the invention is to use the same on-site manufactured yeast in the fermenters, optimized to ferment the combined 1G sugars and the 2G sugars, i.e., the C6 sugars from 1G processes along with combined C5 and C6 sugars from 2G processes, simultaneously. This can increase the effective yield from a 2G process and make it more viable. The on site produced yeast that is being utilized will also convert a portion of the first gen fibers to ethanol.
h. One advantage of systems and methods in accordance with the invention includes utilizing all or at least most of the current equipment in an existing 1G ethanol plant. To the 1G plant, all that can be required is installing the pretreatment and hydrolysis sections of the 2G plant along with the onsite yeast and enzyme production areas. Operating the plant to maximize operating the second gen area at max throughput and reducing the throughput of the 1G area is preferred to stay within the limits of the currently installed distillation area.
i. The corn stover is already available near existing corn ethanol plants.
   The basic process steps for ethanol production from corn in a 1G plant include:
Corn receiving
Corn storage and cleaning
Corn milling.
Mash conversion.
Fermentation.
Carbon dioxide scrubbing
Caron dioxide recovery
Fuel ethanol distillation and dehydration.
Fuel ethanol product storage.
Stillage separation.
Stillage evaporation.
Drying
Dried Distillers Grains with Solubles (DDGS) storage.
Chemical storage.
Clean-in-Place (CIP) system.
Gasoline denaturant offloading and storage.
Truck, barge, or rail ethanol loading facilities.
Truck, barge, or rail DDGS loading facilities.

The basic utilities that can be required include:
Steam generation and condensate return.
Raw water treatment.
Instrument/plant air.
Cooling and chilled water.
Natural gas.
Power distribution.
Fire protection.

The basic process steps for ethanol production from corn stover in a second generation (2G) plant can include:
Feedstock Biomass Corn stover receiving
Corn stover storage
Cleaning and Size reduction.
Pretreatment area
Hydrolysis
Enzyme production area
Yeast Production area.
Lignin Filtration area
Fermentation.
Carbon dioxide scrubbing
Carbon dioxide recovery
Fuel ethanol distillation and dehydration.
Fuel ethanol product storage.
Stillage evaporation.
Lignin Biomass Boiler
Chemical storage.
Clean-in-Place (CIP) system.
Gasoline denaturant offloading and storage.
Truck, rail ethanol loading facilities.

The basic utilities that can be required include:
Steam generation and condensate return. (Biomass Lignin Boiler)
Raw water treatment.
Instrument/plant air.
Cooling and chilled water.
Natural gas.
Power distribution.
Fire protection
   As can be seen the first generation and second-generation equipment requirements are common from the Fermentation area forward. The significant difference is the areas specific for the type of feedstock.

### Process Description for Corn Ethanol Production

### Plant Summary

One non-limiting example of a combination plant in accordance with preferred embodiments of the invention is shown generally as a combination plant 10 in FIG. 1. Combination plant 10 includes a high starch feedstock section 100 modeled after a 1G ethanol plant and process. Corn or another high starch feedstock 110 is typically delivered by truck or rail and stored in steel bins. The corn is typically cleaned using a coarse scalper to remove oversized and foreign material and then the corn is ground to a meal in, e.g., hammer mills. The meal is wetted and mixed with water to form a slurry in a slurry tank 120. The corn starch is then converted to fermentable sugars 136 by the action of known enzymes 135, which are added to slurry from tank 120 in a liquefaction area 130.

The sugars 136 are then sent to a fermentation area 140, where the sugars are converted to ethanol and carbon dioxide by the action of yeast. The yeast component can be optimized to ferment both C5 and C6 sugars in the same fermenter. Fiber enzymes 141 can be added to help convert remaining fiber to additional fermentable sugars. The carbon dioxide can be scrubbed with water to recover trace amounts of ethanol. The carbon dioxide is then vented to the atmosphere or collected for further processing.

A beer 145 from the fermentation tanks is sent to distillation columns 150. The ethanol 151 is recovered from the fermented beer by distillation columns 150 and concentrated to approximately 95% v/v ethanol. This mixture of water and ethanol 151 is an azeotrope and cannot be further separated using standard distillation. The ethanol 151 can then be dehydrated in a molecular sieve dehydration area 160 to produce fuel grade ethanol 170 and transferred to a storage and shipping area.

Residue from the bottom of the beer distillation columns 150, as whole stillage 155 contains valuable nutrients that can be recovered and sold as a high protein animal feed ingredient such as distiller's dried grains with solubles (DDGS). The whole stillage 155 can be decanted 156. Liquid 156a can be sent to an evaporator 152. Solids 156b can be centrifuged to further remove liquids and sent to a DDGS dryer 157. to form DDGS 158. The liquid from evaporator 152 (thin stillage), can be partly recycled to the front end of the process as backset, and can be concentrated by evaporation to provide valuable products such as oils 159.

The evaporator 152 can be partially heat-integrated with the plant and produces syrup with a solids content of about 40%. The centrifuge cake and the syrup can be mixed and fed to a gas-fired dryer system (not shown). Distiller's dried grains with solubles (DDGS) are the nutrient rich co-product of dry-milled ethanol production. DDGS utilization as a feed ingredient is well documented as both an energy and a protein supplement. The dryer 157 removes residual moisture and can produce DDGS with 90% dry matter. The DDGS can then be transferred to a storage area where it can be loaded on trucks or rail cars.

The following descriptions of the individual process steps highlight valuable features of processes designed in accordance with the invention. Key areas include:
Corn Cleaning and Milling
Liquefaction
Yeast Propagation and Fermentation
Distillation
Dehydration
Stillage Evaporators
Stillage Separation
DDGS Dryer
CIP System

### Area—Corn Cleaning and Milling

Corn Cleaning

The corn can be cleaned on the way from storage to processing. This equipment can be sized to clean at the same rate as the milling facility. The cleaning process can include coarse scalping using a screen. Oversize material can be routed to a trash bin.

Corn Milling

In order to be able to gelatinize the starch completely and achieve proper separation in the stillage separation area, the grain should be milled to a specific particle size distribution. Particles that are too coarse can reduce starch conversion to ethanol and lead to plugging of equipment and lines. Very fine particles may not be removed in the decanter and can lead to high syrup viscosity. The milling system should be designed based on the selected particle size distribution and capacity requirements.

The corn can be conveyed and elevated from the storage silos to the coarse scalper. The coarse scalper will gravity discharge the cleaned corn into a surge bin, sized to hold approximately e.g., 2 hours of capacity, above the hammer mills. From the surge bin, the corn will gravity flow into rotary feeders that deliver the corn into the hammer mills.

Hammermills equipped with rotary feeders and magnets are preferably used to grind the corn to meal. The hammer mills discharge into a divided air plenum coupled to a hopper that transitions to a collecting screw conveyor. A rotary airlock at the discharge of the screw conveyor is used to provide an air seal to prevent airflow from the downstream equipment and to facilitate the flow of air through the hammer mill screens. The grinding system equipment can be installed in an open steel structure.

A conveyor and bucket elevator can be used to transfer the meal to a continuous weighing auger system located in the main process building, to measure the rate of meal addition to the mashing process.

Liquefaction

The meal is preferably mixed with hot process condensate in the slurry mix tank to form mash. The mix tank is maintained at a temperature of approximately 175-190° F., preferably about 185° F. Alpha-amylase enzyme can be added to reduce the viscosity so that the mixture can flow more freely. Aqueous ammonia can be added to control pH and to provide a source of nitrogen for yeast nutrition.

The mash can then pumped to the first of two liquefaction tanks, where the starch is hydrolyzed into dextrin by the action of the alpha-amylase enzyme. The two liquefaction tanks can be configured in series with each tank divided into multiple segments to simulate several reactors in series. After liquefaction, the mash is cooled in e.g., a series of heat exchangers and fed to the fermentation tanks. All tanks in this area can be provided with spray machines for routine cleaning via the Clean-in-Place (CIP) system.

### Yeast Propagation and Fermentation

The fermentation process can use tanks that are typically operated in a batch mode. The first tank is used for yeast propagation, where yeast is grown rapidly with the addition of a small amount of air. This tank can be outfitted with a circulation pump and cooler. The tank can also be outfitted with a top-mounted agitator.

Fermentation is preferably accomplished in tanks all of equal size. The fermentation process generates heat, which can be removed by circulating the fermenting mash through external heat exchangers. The fermenters are piped to circulation pumps and coolers for cooling and transferring the beer. These exchangers are preferably plate-and-frame type, designed to minimize plugging between regularly scheduled cleanings.

From fermentation, the beer is pumped to the beer well, a holding tank that allows the beer to be continuously fed to the distillation system regardless of the fermentation mode of operation. To recover energy, the beer can be preheated in a series of heat exchangers using the incoming mash to the fermenters.

The carbon dioxide that is produced during fermentation can be collected and routed to a scrubber. Residual ethanol can be recovered by the scrubber and the resulting carbon dioxide gas is vented to the atmosphere or collected.

Properly adjusting the operating conditions and routine cleaning procedures can minimize the rate of contamination by bacteria. Each fermenter, their pumps, and heat exchangers should be connected to the CIP system for regular cleaning and sterilization. The fermentation system is preferably equipped with piping and controls that allow the system to bypass any fermenter for cleaning or maintenance, as required, while maintaining production without interruption.

### Distillation

The ethanol in the beer can be separated from the stillage using a distillation system. The columns include the beer column and the rectifier column.

After preheating, the beer is pumped to the beer column. The beer is fed in the upper sections of the column where it is stripped of ethanol. The bottoms product of the beer column, or whole stillage, can be routed to an evaporation system. The ethanol vapor from the top of the beer column should be condensed and pumped to the rectifier column where it can be concentrated to near the azeotropic point (95.5% v/v). The concentrated ethanol is then fed to the dehydration system. Fusel oils are typically removed from the rectification section of the column via a vapor draw and mixed with the overheads prior to entering the molecular sieve. The clean rectifier bottoms with minimal ethanol and volatile organics are returned to the front-end of the process.

### Dehydration

The final removal of water from the ethanol to produce fuel grade ethanol can be achieved in a molecular sieve dehydration system. The molecular sieves work on the principle of selective adsorption in the vapor phase. In this case, water is adsorbed on the sieve bed material, while ethanol passes through the bed. The adsorbed water is removed during a regeneration step and is routed back to the distillation system.

Regeneration is achieved using a “pressure swing” system that requires virtually no external heat. The pressure swing is achieved with a vacuum system. Adsorption takes place under positive pressure, while regeneration is accomplished under vacuum.

### Stillage Evaporators

The stillage evaporator system is designed to process the concentrate (thin stillage) from the centrifuges (excluding the thin stillage recycled to the mashing system as backset) and produce a syrup with 40% dry matter concentration. Under conditions where there is no backset being fed to the mashing system, the evaporator system can have the capacity to process all of the thin stillage from the decanters and produce a syrup of reduced solids concentration.

The syrup is stored in the syrup tank and then pumped to the wet cake mixer prior to entering the DDGS dryer. The thin stillage and syrup tanks can provide sufficient surge capacity so that the distillation system can continue to operate at the design rate during regular CIP cleaning of the evaporator system.

### Stillage Separation

The whole stillage from the bottom of the beer column can be routed to decanter centrifuges. The centrifuges should be able to remove more than 60-80%, preferably 78% or more of the suspended solids and produce a cake with approximately 30-40%, preferably about 35% w/w total dry matter concentration, and a liquid concentrate (thin stillage) with about 8-15%, preferably about 11% w/w total solids dry matter. The whole stillage tank should provide sufficient residence time so that the distillation system can continue to operate at the design rate during regular maintenance of the centrifuge system.

### DDGS Dryer

The DDGS dryer system is designed to dry up to 100% of the wet cake and syrup that is produced by the plant at design conditions. The system will typically produce DDGS with about 90% w/w total solids dry matter content.

The dryer system should be designed to meet the emission requirements as defined by the approved permitting documents and the appropriate authority's environmental regulations.

The vapors from the dryer will contain varying amounts of pollutants that should be controlled to meet the requirements of the local, state and federal environmental laws. The most common pollutants requiring control will include particulates (PM/PM10), carbon monoxide (CO), nitrogen oxides (NOx), sulfur oxides (SOx), and volatile organic compounds (VOC).

### CIP System

In order to keep the process microbiologically clean and to remove residues from heat exchange equipment, tanks, and evaporators, a clean-in-place (CIP) system is provided. Caustic is used as a cleaning agent for sanitizing and dissolving most of the residues.

A caustic tank and a process condensate tank are provided. The process condensate tank collects condensate from the evaporators and the distillation system.

The system includes flushing and CIP pipes to all heat exchangers and tanks that require cleaning. Each tank can be equipped with a permanently installed spray machine. Manual and automated isolation valves at each piece of equipment require the operator to set the valves properly before beginning the CIP procedure. Piping is included to allow flushing equipment with process condensate prior to beginning the caustic cleaning cycle.

### Non Process Areas Description

The following summarizes the sections for the off-site process areas:
Grain Receiving and Storage
DDGS Storage and Shipping
Fuel Ethanol Storage and Shipping
Chemical Storage

### Grain Receiving and Storage

Grain receiving and storage is designed to off loads trucks of raw material corn and convey to on site storage silos.

### DDGS Storage and Shipping

DDGS can be mechanically conveyed from the dryer system to the DDGS flat storage building. Distribution conveyors within the building can deposit the material in multiple piles on a flat slab on grade.

A wet cake pad can be constructed outside the main process building. This pad, a flat slab at grade with 3 sides, will be used to hold wet cake from the centrifuge should the dryer be down for maintenance. The wet cake will be manually recycled to the dryer or loaded into trucks. The wet cake pad is sized approximately for 14 hours hold up at design rate.

### Fuel Ethanol Storage and Loading

Fuel ethanol can be pumped to one of the two-day tanks, each sized for e.g., 12 hours of production at design rate. The production rate of the ethanol from the distillation/dehydration system should be monitored. Moisture content can be monitored with laboratory equipment from regularly scheduled samples. After the quality of the ethanol is confirmed, it is transferred to one of the two product storage tanks. Each storage tank can be sized to hold e.g., approximately 4.3 days of 100% production rates. An approved production meter will be supplied to gauge the transfer of ethanol into the storage tank.

If the ethanol does not meet specification, it should either be transferred back to the process for further purification or transferred to the product storage tanks to be blended with on-spec ethanol to create an on-spec mixture.

A blending system can be used to blend gasoline denaturant (at up to 5% v/v) from a denaturant storage tank into the ethanol as it is transferred to one of the product storage tanks.

### Chemical Storage

Selected high use chemicals can be stored on site in bulk and distributed on site through dedicated piping systems.

### Utilities

The following descriptions summarize the basic sections for the utility areas:
Cooling Water
Steam and Steam Condensate
Water Supply and Distribution
Compressed Air
Fire Protection
Natural Gas
Electrical Supply and Distribution
Waste Water Treatment
Process Effluent
Sanitary Sewage
Storm Water
Solid Waste Disposal

### Cooling Water

An induced draft cooling tower should be provided.

The cooling water circulating capacity and cooling requirements for the processes in accordance with the invention should be designed with respect to parameters known by those in the industry. The ASHRAE summer 1% frequency for maximum wet bulb temperature can be used for the design basis. One spare cooling water pump should be provided.

A chilled water refrigeration unit can be provided to cool the fermentation system during those periods when the cooling water temperature from the cooling tower exceeds 70° F.

One spare chiller pump can be provided and sized to convey 50% cooling water flow.

Steam and Steam Condensate

Steam can be provided by boiler units fueled with natural gas and/or lignin. The steam and condensate return requirements and conditions for the process can be calculated by those of skill in the art in accordance with known.

### Water Supply and Distribution

The configuration for this system will depend on local conditions and will be designed along known parameters.

### Compressed Air

Process air requirements for yeast propagation and fermentation and instrument air requirements will be set along normal, known parameters. A separate compressor can be used for instrument air and process air.

### Fire Protection

Configuration for this system should be based on the local requirements.

### Natural Gas

Configuration for this system should be based on local requirements.

### Electrical Supply and Distribution

The connection to the local can be local to the plant site.

### Back-Up Power

A 4500 KW back-up generator should be provided. Alternated sizes can be substituted as needed.

### Waste Water Treatment

Waste water treatment is typically not required for process purposes. Local ordinances should be followed.

### Process Effluent

All effluent generated by the process units, over and above what is directly recycled to the process, can be directed to a water collection tank configured to discharged, e.g., to a river and may require additional processing as needed. Material from the water collection tank can be recycled into the process at an appropriate flow rate. Boiler blow down can be routed to the dryer. Cooling tower blow down post treatment can be routed to configured discharge system.

### Sanitary Sewage

Sanitary sewer services such as a septic system will entail staff washrooms in the administration and the main process building designed for approximately 40 people. Configuration for this system will be local regulations.

### Storm Water

Configuration for this system should comply with local regulations.

### Solid Waste Disposal

Configuration for this system should comply with local regulations.

### Process Water Treatment

A process water treatment system may be required for boiler make-up depending on a site specific water analysis.

### Process Description for the Cellulosic Feedstock System

Combination plant 10 also includes a cellulosic feedstock section 200 modeled after a 2G ethanol plant and process. 2G processes are known for converting cellulosic, feedstocks, such as corn stover and other agricultural waste into liquid fuels. One particularly well suited process is the Sunliquid® process developed and implemented by Clariant in Germany. The Sunliquid® process developed by Clariant was developed to meet all the requirements of a technically and economically efficient, innovative process for converting agricultural residues into biofuel. Using process-integrated enzyme production, optimized enzymes, simultaneous conversion of cellulose and hemicellulose into ethanol and an energy-efficient process design, this process can overcome technological challenges and sufficiently reduce production costs in order to arrive at a commercially viable basis.

### Exemplary Raw Material/Mechanical Pretreatment (e.g., Clariant Sunliquid®)

Cellulosic materials receiving section 200 includes a receiving, storage and milling of the corn stover 210. As a base exemplary case, bales with a weight of 500 kg/bale and the following exemplary approximate dimensions can be used: L=2,400 mm, W=1,200 mm, H=900 mm.

The main storage of the stover “straw” can be either centralized in the vicinity of the plant or it can be in decentralized locations preferably within e.g., 75 to 100 km from the plant. The straw from decentralized locations is preferably delivered to the plant “just-in-time” by truck. However, to guarantee a continuous supply of straw to the process, a buffer storage of 1-2 days production i.e. approximately 2000 tons of straw is advantageous. Furthermore, an outside storage area for approx. another 3-4 days storage is normally employed to secure the supply of the straw to the process during e.g. road blocks, inclement weather etc. The straw bales are transported from the storage area to the straw mill, which is preferably operated continuously and cuts the straw to an average length of approx. 5 cm. Before the mill, an automatic string cutter removes the strings attached to the bales. Next, the straw is milled and passes through a system of detectors to remove metals, stones, dust, etc. The straw chaff is then transported for thermal pretreatment.

### Thermal Pretreatment

A thermal pretreatment 220 is important to further breakdown the straw to make the cellulose and hemicellulose more accessible for enzymes in the saccharification process. The pretreatment can be done in a 1-stage pretreatment reactor, design and performance validated in Pulp and Paper industry on commercial scale. No chemicals (i.e. acid or lye) need be used in this process step. A feed of The straw chaff 210 enters a reactor 220 and steam is directly injected. Due to the heat, pressure and retention time, the straw chaff 220 is broken down and a so called pretreated “substrate” 225 is obtained. The substrate 225 exits the pretreatment reactor 220 through a blow line and a pressure drop to atmospheric pressure takes place. Next, the steam is separated from the solid substrate 225 and transported to the hydrolysis vessels 230. Some or all of the steam can be recovered and further used in the process.

### Enzymatic Hydrolysis

In the enzymatic hydrolysis 230, the pretreated material is converted to C6 and C5 sugars using enzymes. The enzymatic hydrolysis of the substrate 225 can be done in several parallel stirred tank reactors operated in batch mode, while substrate feeding and product discharge can be done continuously. One batch consist of filling, reaction, emptying and cleaning if necessary. The inputs in the batch for hydrolysis are enzymes 231 from enzyme production 235, substrate 225 from thermal pretreatment and process water. The mash in the hydrolysis vessels 230 is continuously agitated to ensure homogeneous reaction conditions. At the end of the reaction a suspension of solid by-product lignin in an aqueous sugar rich solution called “hydrolysate” 238 is present.

Post the reaction, the hydrolysate is pumped to a lignin filtration area 240.

### Lignin Filtration

After hydrolysis, the lignin present in the hydrolysate 238 should be separated. By optimizing the sequence protocol, a dry matter >50%, even 60% of the lignin filter is typically obtained for known feedstocks. The discharged lignin 245 is collected and transported e.g. to an attached power plant or lignin boiler 249. The lignin-free hydrolysate 248 is sent to the ethanol fermentation unit 140 of process line 100.

### Fermentation

The conversion of C6 and C5 sugars from 2G line 200 into ethanol is carried out in the ethanol fermentation unit 140. The fermentation unit 140 includes a yeast buffer tank, main fermenters and a mash tank. The temperature in the fermenters is maintained to ensure a stable ethanol production. The filtrate 248 containing C5 and C6 sugars is pumped into the main fermenter along with the yeast from the yeast buffer tank to start the fermentation of sugars into ethanol. The mash solution is transferred to the ethanol purification unit. The exhaust air from fermentation can be led to an off-gas scrubber where ethanol is recovered and fed back to the mash tank. After fermentation, a thorough cleaning procedure should be employed to ensure stable process conditions.

### Ethanol Purification

The ethanol purification unit comprises 3 process stages:
Beer column
Rectification
Ethanol dehydration

The purification unit should be equipped with a modern, energy-saving multi-stage distillation.

The mash solution first enters the mash column, where alcohol is concentrated and sent as “raw ethanol” to the rectification stage. Non-condensable gases are separated and should be treated in a gas scrubber before being blown off to the atmosphere. The rectification further concentrates the ethanol stream to approx. 95% and purifies it from side-products which are removed as heads and feints and fusel oils. In the ethanol dehydration stage, the final bioethanol product quality of over 99%, preferably over 99.5%, most preferably about 99.8% and approaching 100% is achieved by removing the remaining water with molecular sieves. The bioethanol is cooled and send to the tank farm.

### Vinasse Evaporator

Vinasse (water and solids byproduct) produced in the ethanol purification unit can be concentrated in a multiple-effect evaporator to a dry matter content of typically about 60%. Heat is provided by steam. The condensate fraction can be recycled back to the process as process water. The obtained concentrated vinasse should be stored in buffer tanks.

### Ethanol Storage Tank Farm

The quality of ethanol should be monitored continuously in the transfer line by e.g., a density measurement. If it does not meet the prescribed quality, the ethanol can be pumped back to the rectification column. The bioethanol quality is preferably controlled daily in the bioethanol day tank and pumped to product tank, if the product meets the specifications. Depending on the site location, train, truck or barge loading systems are foreseen.

Tanks for side-products of the purification process (e.g. fusel oils, heads and feints) are also located in this area.

### Enzyme Production

Enzymes required for the enzymatic hydrolysis can be produced in a dedicated separate section of the plant, thus eliminating the need for transportation, formulation and logistics. The carbon source needed for the microorganisms to produce enzymes can be a small fraction of the pretreated lignocellulosic material.

The enzyme production can include parallel fermentation cascades. Each cascade is composed of pre-fermenters and end-fermenters for enzyme production. The pH value of the process can be controlled by using acid and lye ammonia respectively. The fermenters should be equipped with agitators for homogenized reaction conditions and dispersion of air.

The produced enzymes are directly sent to the enzymatic hydrolysis unit.

### Yeast Production

Yeast required for the ethanol fermentation can be produced in similar vessels as the enzyme production unit. Sugar containing hydrolysate from the enzymatic hydrolysis unit can be used as a nutrient solution for the yeast propagation. The yeast production comprises a fermentation cascade composed of consecutive yeast pre-fermenters and yeast end-fermenter. The pH value of the process can be controlled by using acid and lye respectively. The fermenters can be equipped with agitators for homogenized reaction conditions and dispersion of air.

The produced yeast can be sent directly to the ethanol fermentation unit.

### Supporting Processes

The following units for utilities and infrastructure are important to support the cellulosic production line 200 process. These systems are known in the industry and are similar to those in 1G corn ethanol facilities.
Cooling Tower: can be an evaporative cooling tower system.
Cold Water: Cold Water Chillers to be designed for heat load especially in Enzyme and Yeast Production areas
Chemical Storage: all equipment necessary for storing the required amounts of chemicals needed to operate the plant.
Process Air: for process air requirements. yeast and enzyme production require process air.
Waste Water collection: waste water from the various units of the plant.
Intermediate Product Storage: Production day tanks for ethanol product, off spec ethanol product, fusel oil, heads and feints and furfural.
Other Utilities: CIP, Additives
   The following OSBL process units for utilities and infrastructure are required to support the 2G process. These systems are known in the industry and are similar to those in 1G corn ethanol facilities.
Boiler/Co-Generation Plant: a solid fuel boiler is provided to burn the lignin by-product generated by the process.
Raw Water Treatment: A raw water treatment system may be required dependent on the quality of water make-up to the facility.
Ethanol Storage and Load Out: tankage and equipment required to enable the shipment of ethanol off site.
Instrument Air:
Waste Water Treatment Plant
Final product Storage and Loadout
Vinasse Utilization:

### Rin Background Information

1. To both enforce the mandate for their use and develop the markets for these fuels, the EPA developed a trading and enforcement program comprised of obligated parties and renewable producers. Obligated parties are refiners or importers of gasoline or diesel fuel, and are given the name “obligated” as they are required to comply by blending renewable fuels into transportation fuel, or by obtaining credits called RINs to meet an EPA-specified volume obligation—the Renewable Volume Obligations or RVOs. Obligated parties are to demonstrate compliance with the program by the end of the compliance year, but are given flexibility in carrying a compliance deficit into the next year. That deficit must be made up by the end of the extended year.
2. This program was authorized under the Energy Policy Act of 2005 and expanded, often called the RFS2, under the Energy Independence and Security Act of 2007. Key expansion items included higher long-term goals to more than double current volumes, calendar extension to 2022, grandfathering of certain original fuels, explicit defining of qualifying fuels, and introduction of specific waiver authorities. Renewable fuels fall into four categories, each with their own annual volume standards shown in FIG. 1:
   Cellulosic Biofuel (D3): Produced from cellulose, hemicellulose, or lignin and must meet a 60 percent lifecycle GHG reduction.
   Advanced Biofuel (D5): Produced from a non-corn starch, renewable biomass and must meet a 50 percent lifecycle GHG reduction.
   Biodiesel (D4): Must be biomass-based diesel and meet a 50 percent lifecycle GHG reduction.
   Corn-Based Ethanol (D6): Ethanol derived from corn starch and must meet a 20 percent lifecycle GHG reduction.
3. RINs (or rins) are the backbone of the program. They are the currency used in trading. Every equivalent gallon of renewable fuels is assigned a RIN at its point of generation or origination. These RINs work much like Renewable Energy Credits (RECs) work in the generation and trading of renewable electricity. RINs can be traded between parties, bought as attached RINs to fuel purchased, and/or bought unattached on the open market. Unlike California's Low Carbon Fuel Standards (LCFS) program, RINs expire. Unused RINs can carry over via the additional extended compliance year allowance, but beyond that, if not used, they expire. This expiration element is why RINs are traded with not only its RIN classification (next section), but also with its generation and therefore expiration date.
4. Recognizing short-term difficulty in attaining required volumes of cellulosic standards, EPA has added even more flexibility to the program beyond that generated by the nesting principle. This is the cellulosic waiver credit (CWC), which is offered by EPA, at a price determined by formula in the statute, so that obligated parties have the option of purchasing CWCs plus an advanced RIN in lieu of blending cellulosic or obtaining a cellulosic RIN. The published price of a CWC in 2017 by EPA was $2.00. The trading theory is that the value of a D3 RIN should be fairly close to the value of a D5 RIN plus the CWC.
5. EPA has determined that each type of RIN must be compared to another through a comparison of its fuel value per unit volume to that of pure liquid ethanol fuel. Each gallon of ethanol has about 77,000 Btu (or 0.077 MMBTU), which is thus the definition of a RIN. A useful conversion to study the business case for RINs is to convert the value of the credits to dollars per MMBtu. Here's the math:
6. $ X/RIN×RIN/0.077 MMBTU=$ X/MMBTU
   Those skilled in the art, taking into account the various embodiments of the ethanol production systems and methods described herein and the principles of operation of the same, by employing routine experimental procedures can readily optimize the design of a particular system and method in accordance with the present teachings.

The present teachings encompass embodiments in other specific forms without departing from the spirit or essential characteristics thereof. The foregoing embodiments are therefore to be considered in all respects illustrative rather than limiting on the present teachings described herein. Scope of the present invention is thus indicated by the appended claims rather than by the foregoing description, and all changes that come within the meaning and range of equivalency of the claims are intended to be embraced therein.

What is claimed is:

1. A method for manufacturing ethanol, comprising:
in a first ethanol production line, a corn feedstock is milled, subjected to saccharification, and starches of the milled corn are converted into corn sourced fermentable sugars;
in a second ethanol production line, separate from the first ethanol production line, corn stover feedstock is milled, subjected to saccharification and the corn stover feedstock is converted into fermentable stover sourced sugars;
the corn sourced sugars and the stover sourced sugars are fed to a common fermentation tank and subjected to fermentation to produce ethanol and carbon dioxide.

2. The method of claim 1, wherein the ethanol is distilled to at least about 95% ethanol.

3. The method of claim 2, wherein the ethanol is purified to over about 98%, ethanol, by use of molecular sieve.

4. The s method of claim 1, wherein prior to saccharification, the milled corn stover is subjected to a pretreatment process step including heat to disrupt the fiber structure of the corn stover feedstock to make it more accessible to saccharification enzymes.

5. The method of claim 4, wherein after the pretreatment step, the pre-treated corn stover feedstock is subjected to hydrolysis to produce a hydrolysate feed.

6. The method of claim 5, wherein the method of manufacturing ethanol requires the production of steam, the hydrolysate feed comprises lignin and after the hydrolysis step, at least some of the lignin is removed from the hydrolysate feed and at least some of the removed lignin is burned on-site to satisfy some of the steam requirements for the method of manufacturing ethanol.

7. The method of claim 6, wherein the ethanol produced by fermentation is distilled to increase the ethanol percentage and the removed lignin is burned to supply at least some of the energy for the distillation of the ethanol.

8. The system and method of claim 1, wherein the first ethanol production line operates within an existing ethanol production facility designed to produce ethanol from corn and the second ethanol production line is added to the existing ethanol production facility.

9. A facility constructed to manufacture ethanol, comprising:
a first ethanol production line constructed and arranged to process a corn feedstock comprising corn starch, including a corn mill and a saccharification tank, the corn mill adapted to reduce the particle size of the corn and provide the reduced size corn to the saccharification tank, which is adapted to convert the corn starch to a corn sourced sugar and provide the corn sourced sugar to a fermentation tank;
a second ethanol production line, separate from the first ethanol production line, constructed and arranged to process corn stover feedstock, comprising a mill and a saccharification tank, the mill adapted to reduce the size of the corn stover, and provide the reduced size corn stover to the saccharification tank, which is adapted to convert the reduced size corn stover into stover sourced sugar and provide the stover sourced sugar to the fermentation tank;
the fermentation tank including an effective amount of a yeast component to ferment at least some of the corn sourced sugar and the stover sourced sugar, the fermentation tank adapted to ferment sugar into ethanol and carbon dioxide.

10. The facility of claim 9, and comprising a distillation section coupled to the fermentation tank, adapted to receive the ethanol from the fermentation tank and distill the ethanol to a higher purity ethanol.

11. The facility of claim 10, wherein the second production line comprises and a pretreatment tank coupled to and located downstream from the corn stover mill, adapted to receive and subject the reduced size corn stover to a pretreatment step including heat to disrupt the fiber structure of the corn stover feedstock and make it more receptive to saccharification enzymes.

12. The facility of claim 11, wherein the second production line comprises a hydrolysis tank coupled to and located downstream from the pretreatment tank, adapted to subject the pre-treated corn stover to hydrolysis to produce a hydrolysate feed.

13. The facility of claim 12, wherein the second production line comprises a lignin separator coupled to and located downstream from the hydrolysis tank, adapted to separate and remove at least a portion of any lignin in the hydrolysate and direct the lignin-reduced hydrolysate to the fermentation tank.

14. The facility of claim 13, and comprising a lignin boiler, coupled to and located downstream from the lignin separator, adapted to burn the lignin separated from the hydrolysate and produce steam for use in the facility.

15. The facility of claim 14, wherein the fermentation tank is adapted to receive and combine corn sourced sugar from the liquefaction device and stover sourced sugar from the lignin separator.

16. The facility of claim 14, wherein the lignin boiler is adapted and located to produce steam for the distillation of the ethanol.

17. The facility of claim 9, wherein the yeast component is selected to ferment both C5 and C6 sugars in the same fermentation tank.
